(19) United States
(12) Patent Application Publication
(10) Pub. No.: US 2025/0085262 A1
(43) Pub. Date: Mar. 13, 2025

(54) METHOD AND APPARATUS TO MEASURE HYDROGEN SULFIDE CONCENTRATION IN A GAS MIXTURE WITH GAMMA RAY ABSORPTION

(71) Applicant: Schlumberger Technology Corporation, Sugar Land, TX (US)

(72) Inventors: Sukru Sarac, Dharan (SA); Diogo Piasseski, Dharan (SA); Yue Sum Wilson Chin, Singapore (SG); Guillaume Jolivet, Clamart (FR)

(21) Appl. No.: 18/720,395

(22) PCT Filed: Dec. 21, 2022
(86) PCT No.: PCT/US2022/053674

§ 371 Date: Jun. 14, 2024

Related U.S. Application Data
(60) Provisional application No. 63/265,794, filed on Dec. 21, 2021.

(51) Int. Cl. G01N 33/00; G01N 23/06

(52) U.S. Cl. CPC G01N 33/0044; G01N 23/06

(57) ABSTRACT

Embodiments described herein provide methods and apparatuses to determine hydrogen sulfide (and carbon dioxide) concentration in a gas mixture using gamma photon absorption analysis.

### CROSS-REFERENCE TO RELATED APPLICATIONS

This application for patent claims benefit of U.S. Provisional Patent Application Ser. No. 63/265,794 filed Dec. 21, 2021, which is entirely incorporated herein by reference.

### BACKGROUND

This disclosure relates generally to hydrocarbon production and, more particularly, to methods and apparatus for hydrogen sulfide concentration measurement in a flowing gas mixture.

### DESCRIPTION OF THE RELATED ART

Most oil-gas wells produce a mixture of oil, water, and gas. During hydrocarbon production, a determination of flow rates of individual phases (e.g., oil, gas, water, etc.) of a multiphase flow is desirable. The individual phase flow rates can be derived from the measured phase volume fractions and phase flow velocities. A determination of other properties of the gas phase mixture is also desirable, including the presence and concentration of hydrogen sulfide and carbon dioxide in the gas phase. Such properties can be used to determine information about the gas mixture and may affect other measurements being made on the multiphase mixture.

The inline or online monitoring and detection of hydrogen sulfide (H₂S) is a long-standing issue in the oil and gas industry. Hydrogen sulfide is not only highly corrosive to equipment but is also toxic at concentrations of 10 ppm (part-per-million) or more and lethal at concentrations above 500 ppm. Unfortunately, H₂S may exist naturally in oil and gas wells up to a concentration of 50% which poses significant environmental and safety hazard. Thus, it is desirable for safety risk management to have a reliable inline H₂S monitoring device. The use of gamma ray absorption to measure inline or online the concentration of one or two gases simultaneously in a gas mixture, for example, the concentration of H₂S and/or CO₂ in a hydrocarbon gas mixture would thus be desirable. The method applies in general to any two arbitrary gases in a composite gas mixture. Conventionally, gas samples are collected from wells and are analyzed in laboratories to measure their composition; however, because H₂S is very reactive, losses can occur during sample storage and transportation to the laboratories. Hence, on-site measurements are commonly used. Conventional surface well testing to date uses stain tubes to sample and measure both H₂S and CO₂, which requires a gas sample that is normally drained from the stream to the atmosphere. Embodiments of the present disclosure eliminate the need for regular containment breaking and gas release to the atmosphere.

### SUMMARY

Certain aspects of some embodiments disclosed herein are set forth below. It should be understood that these aspects are presented merely to provide the reader with a brief summary of certain embodiments and that these aspects are not intended to limit the scope of the subject matter described herein.

Embodiments described herein provide methods and apparatuses of determining the presence of hydrogen sulfide in a flowing gas mixture. The method includes passing electromagnetic radiation of at least one energy level through a gas phase of a multiphase fluid, measuring the absorption of the electromagnetic radiation by the gas, and determining the gas concentration of at least one gaseous component in the gas phase based on the absorption of the electromagnetic radiation.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 shows a schematic diagram of a dual-energy gamma ray absorption (DEGRA) setup.

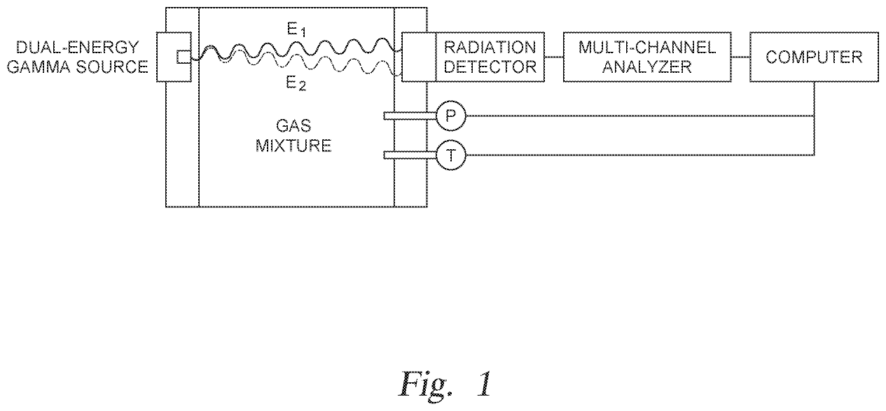

FIG. 2 shows an exemplary linear attenuation triangle for hydrogen sulfide, carbon dioxide and the residual hydrocarbon gas.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Some embodiments of the disclosure describe methods and apparatuses for inline or online hydrogen sulfide (H₂S) and/or carbon dioxide (CO₂) trending and measurements. The method targets the use of MPFM at surface well testing facilities, which have an inherent uncertainty, as the upfront fluid composition is not always known to enable accurate monitoring throughout the operation. The initial proposed application in surface well testing facilities does not limit its use. Given that the incomplete upfront fluid composition has a series of implications which are now bridged with the method proposed, the application of the method thus extends to other production scenarios.

Error! Reference source not found. is a schematic view of a dual-energy gamma ray absorption (DEGRA) apparatus according to one embodiment of the present disclosure. A radioactive source is selected based on its capability of emitting at least two distinct energies of gamma-ray and/or X-ray photons. A radiation detector, such as a scintillator detector is used to detect the photons after they pass through the gas mixture. A multi-channel analyzer or the like is then used to determine the attenuations of the transmitted photons at the two different energies. Finally, a computer is used to evaluate the H₂S and CO₂ concentration based on the photon attenuation, pressure and temperature measurements.

The gas concentration is measured based on absorption of photons, usually gamma photons. The principle of DEGRA has been widely applied in the oil and gas industry to measure the individual water, oil, and gas phase fraction of a well fluid. The same principle, when applied to a gas flow line, can be used to measure, for instance, the individual concentration of H₂S, CO₂, and the residual gas phase.

Gamma ray absorption by a material can be described by the Beer-Lambert Law which is written as

$$N ( E ) = {N}_{0} ( E ) exp [ - ( \vec{α} \cdot \vec{λ} ) d ] \qquad ( 1 )$$

where N(E) is the transmitted photon count rate at energy E, N₀ is the incident photon count rate at energy E, \vec{α} is the phase fraction vector, \vec{Δ} is the linear attenuation coefficient vector, and d is the path length traversed by the beam. If λₘ≡\vec{α}·\vec{λ} is the linear attenuation coefficient of the mixture measured using count rates of gamma photons at two energy levels, i.e. N(E₁) and N(E₂), a system of linear equations can be solved for three independent phases \vec{α}=(α₁, α₂, α₃). The system of linear equations in matrix form is

$$( \begin{matrix}{λ}_{1 , {E}_{1}} & {λ}_{2 , {E}_{1}} & {λ}_{3 , {E}_{1}} \\ {λ}_{1 , {E}_{2}} & {λ}_{2 , {E}_{2}} & {λ}_{3 , {E}_{2}} \\ 1 & 1 & 1\end{matrix} ) ( \begin{matrix}{α}_{1} \\ {α}_{2} \\ {α}_{3}\end{matrix} ) = ( \begin{matrix}{λ}_{m , {E}_{1}} \\ {λ}_{m , {E}_{2}} \\ 1\end{matrix} ) \qquad ( 2 )$$

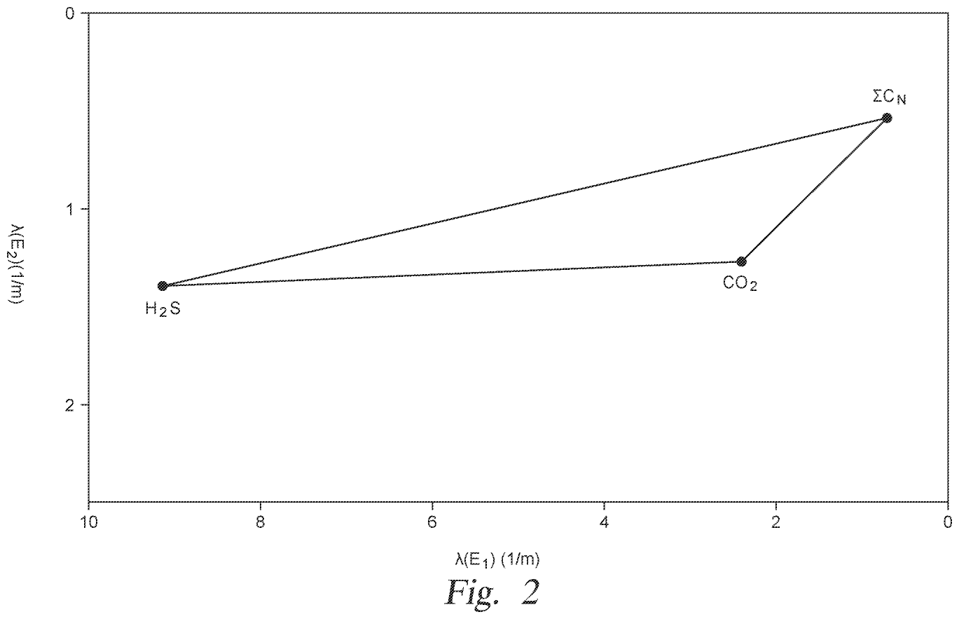

In the context of H₂S measurement in a gas mixture, \vec{α}=(α₁=α_{H}_{₂}_{S}, α₂=α_{CO}_{₂}, α₃=α_{g}). FIG. 2 shows an exemplary corresponding linear attenuation triangle.

Where the concentration of CO₂ is substantially constant and known a priori, the second energy can be omitted to truncate the matrix to avoid an overdetermined system:

$$( \begin{matrix}{λ}_{1 , {E}_{1}} & {λ}_{2 , {E}_{1}} \\ 1 & 1\end{matrix} ) ( \begin{matrix}{α}_{1} \\ {α}_{2}\end{matrix} ) = ( \begin{matrix}{λ}_{m , {E}_{1}} \\ 1\end{matrix} ) \qquad ( 3 )$$

such that \vec{α}=(α=α_{H}_{₂}_{S}, α₂=α_{g}) in which α_{g} accounts for all residual gas including CO₂. Therefore, measuring H₂S concentration in a gas mixture with substantially constant and known CO₂ concentration can be done by measuring absorption of gamma photons at one energy level.

The linear attenuation coefficient is defined as

$$λ = μ ρ \qquad ( 4 )$$

where μ is the mass attenuation coefficient and p is the density. Conventional DEGRA measurement for water, oil, and gas requires a fluids in-situ calibration process that includes measuring absorption of each phase separately, water, oil, and gas. The linear attenuation coefficient of each component, inherently defined by the molecular composition and density of the component, is determined from the measured absorption. Applying DEGRA to H₂S, CO₂, and gas requires a different calibration approach because the individual gas attenuation coefficients cannot be measured in the field.

The linear attenuation coefficients of the gas components can be derived from photon absorption analysis of a representative gas mixture. The mass attenuation coefficient μ of known molecular composition such as H₂S and CO₂ are readily available in National Institute of Standards and Technology (NIST) database. The residual gas is substantially hydrocarbon. The mass attenuation coefficients of different hydrocarbon gases are very similar, so different compositions of hydrocarbon gases result in similar mass attenuations. Therefore, inline H₂S fraction measurement is substantially insensitive to the residual gas composition and a-priori knowledge of the residual gas composition is not necessarily needed. However, to further improve measurement accuracy, the mass attenuation for the residual gas mixture can be determined from a similar gas sample with a representative residual gas composition, for example, as follows:
1. Perform a gas composition analysis on a similar gas sample to obtain the gas composition and concentration. This gas can have different H₂S and CO₂ composition compared to the actual gas sample, in which the H₂S concentration will be measured using methods described herein.

$$\sum {α}_{t o t a l} = \sum {α}_{g a s} + {α}_{{H}_{2} S} + {α}_{{CO}_{2}} = 1 ,$$

where the residual gas Σα_{gas}=α_{CH}_{₄}+α_{C}_{₂}_{H}_{₆}+α_{C}_{₃}_{H}_{₈}+ . . . , includes everything that is not H₂S and CO₂.
2. Renormalize the concentration of residual gas excluding H₂S and CO₂, i.e.

$$\begin{matrix}{α}_{{CH}_{4}}^{′} = \frac{{α}_{C {H}_{4}}}{\sum {α}_{g a s}} \\ \sum {α}_{g a s}^{′} = {α}_{C {H}_{4}}^{′} + {α}_{{C}_{2} {H}_{6}}^{′} + {α}_{{C}_{3} {H}_{8}}^{′} + … = 1\end{matrix}$$

3. The mass attenuation of residual gas mixture is then calculated as

$${μ}_{g a s} ( E ) = {\sum }_{i} {α}_{i}^{′} {μ}_{i} ( E )$$

where μᵢ is the mass attenuation of each gas, which is available in NIST database.

Resolving the linear attenuation coefficient of the gas components depends on determining a density of each component (Equation 4). The density of individual gas components of interest (H₂S, CO₂ and residual gas) can be calculated using an equation of state model with measured pressure and temperature as input parameters. However, determining the apparent density of each component for purposes of Equation 4 is complicated by the fact that real gases do not follow Boyle's law, where the mixture gas density is equal to the weighted average of the individual gas densities, weighted by their fractions in the total gas stream. For example, the apparent density of H₂S, which is needed for the linear attenuation coefficient computation, varies depending on the H₂S concentration in the gas mixture and the composition of the remaining gas, including the CO₂ concentration.

To determine the apparent density of H₂S, an interative computation can be performed. If density of each component is ascertained, composition of the representative gas mixture can be calculated from gamma photon absorption analysis. To determine the apparent density of H₂S, for example, an initial H₂S fraction measurement is performed by gamma photon absorption analysis using photons at two energy levels. Density of the mixture is also computed by fluid property computations using equations of state or black-oil correlations. An initial assumption is made that the apparent density of H₂S in the mixture is the pure H₂S density at the measurement pressure and temperature conditions. Linear attenuation coefficient for H₂S is determined (Equation 4), and then a composition is calculated using the measured gamma photon absorption (Equation 1). The calculated composition is then used to determine an updated apparent density for H₂S using ideal gas law for the gas mixture. Composition and apparent density for H₂S are iteratively calculated until the compositions converge. The same procedure can be used to determine CO₂ apparent density where CO₂ concentration can change substantially. The two apparent densities can be converged simultaneously in one iterative process, or using sequential iterative computations.

While the foregoing is directed to embodiments of the present invention, other and further embodiments of the present disclosure may be devised without departing from the basic scope thereof, and the scope thereof is determined by the claims that follow.

What is claimed is:

1. A method, comprising:
measuring attenuation of gamma photons in a gas mixture consisting essentially of hydrocarbons, CO₂, and H₂S; and
determining composition of the gas mixture using the measured attenuation.

2. The method of claim 1, wherein the gamma photons are photons at two energy levels.

3. The method of claim 2, further comprising determining linear attenuation coefficients for components of the gas by measuring attenuation of the gamma photons at two energy levels in a representative gas of known composition consisting essentially of hydrocarbons, CO₂, and H₂S.

4. The method of claim 1, wherein the gamma photons are photons at one energy level.

5. The method of claim 3, wherein determining the linear attenuation coefficient for the components of the gas comprises computing density of the hydrocarbons in the representative gas using an equation of state model with temperature and pressure as input.

6. The method of claim 3, wherein determining the linear attenuation coefficients for components of the gas comprises calculating a density of the representative gas and determining an apparent density of components of the representative gas from the measured attenuation of the gamma photons by the representative gas, the calculated density, and literature values for mass attenuation coefficients of the components of the gas.

7. The method of claim 6, wherein determining the apparent density of component of the representative gas is performed using an interative computation.

8. The method of claim 3, wherein determining linear attenuation coefficients for components of the gas comprises treating the hydrocarbons in the gas as a single component.

9. A method, comprising:
measuring attenuation of gamma photons in a gas mixture consisting essentially of hydrocarbons, CO₂, and H₂S;
measuring temperature and pressure of the gas mixture; and
determining composition of the gas mixture using the measured attenuation, temperature, and pressure.

10. The method of claim 9, wherein the gamma photons are photons at two energy levels.

11. The method of claim 10, further comprising determining linear attenuation coefficients for components of the gas by measuring attenuation of the gamma photons at two energy levels in a representative gas of known composition consisting essentially of hydrocarbons, CO₂, and H₂S.

12. The method of claim 9, wherein the gamma photons are photons at one energy level.

13. The method of claim 11, wherein determining the linear attenuation coefficient for the components of the gas comprises computing density of the representative gas using an equation of state model with temperature and pressure as input.

14. The method of claim 11, wherein determining the linear attenuation coefficients for components of the gas comprises computing a density of the representative gas and determining an apparent density of components of the representative gas from the measured attenuation of the gamma photons by the representative gas, the computed density, and literature values for mass attenuation coefficients of the components of the gas.

15. The method of claim 14, wherein determining the apparent density of components of the representative gas is performed using an interative computation.

16. The method of claim 11, wherein determining linear attenuation coefficients for components of the gas comprises treating the hydrocarbons in the gas as a single component.

17. A method of determining composition of a mixture of H₂S, CO₂, and hydrocarbons, the method comprising:
measuring attenuation of gamma photons in a gas mixture consisting essentially of hydrocarbons, CO₂, and H₂S;
measuring temperature and pressure of the gas mixture;
determining linear attenuation coefficients for the components of the gas mixture by measuring attenuation of the gamma photons in a representative gas mixture of H₂S, CO₂, and hydrocarbons; and
determining composition of the gas mixture using the measured attenuation and the determined linear attenuation coefficients.

18. The method of claim 17, wherein measuring attenuation of the gamma photons in the gas mixture and in the representative gas mixture uses gamma photons at two energy levels.

19. The method of claim 17, wherein measuring attenuation of the gamma photons in the gas mixture and in the representative gas mixture uses gamma photons at one energy level.

20. The method of claim 17, wherein determining linear attenuation coefficients for the components of the gas mixture comprises iteratively calculating composition of the representative gas mixture based on the measured attenuation of gas photons in the representative gas mixture, measured density of the representative gas mixture, and measured temperature and pressure of the representative gas mixture.
